(19) United States
(12) Patent Application Publication
(10) Pub. No.: US 2001/0016569 A1
(43) Pub. Date: Aug. 23, 2001

(54) Receptor ligands

(75) Inventors: Martin Rosenberg, Royersford, PA (US); Katherine Louisa Widdowson, King of Prussia, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 09/804,852

(22) Filed: Mar. 13, 2001

Related U.S. Application Data
(63) Continuation of application No. 08/963,835, filed on Nov. 4, 1997, now abandoned.
(60) Provisional application No. 60/030,391, filed on Nov. 5, 1996.

(51) Int. Cl. A61K 38/00; A61K 31/55; A61K 31/54; A61K 31/535

(52) U.S. Cl. 514/2; 514/9; 514/217; 514/224.8; 514/229.8

(57) ABSTRACT

Non-antibody multimeric receptor ligands, methods for making and identifying them and their use for agonizing or antagonizing multimeric receptors.

### RELATED APPLICATIONS

This application claims the benefit of U.S. Provisional Application No. 60/030,391, filed Nov. 5, 1996.

### FIELD OF THE INVENTION

This invention relates to multimeric receptor ligands, methods for making and identifying them and their use as agonist or antagonists of multimeric biological receptors.

### BACKGROUND OF THE INVENTION

Many receptors of the single transmembrane class appear to respond to ligand binding by some form of aggregation. Aggregation can be in the form of homodimerization or homotrimerization in the case of identical receptor subunits or in the form of heterodimerization or heterotrimerization in the case of different receptor subunits. It has become clear in several systems that receptor aggregation is part of the signal for the target cell to respond biologically. See review by Young, P. R. entitled “Protein hormones and their receptors” in Curr. Opin. Biotech. 3, 408-421 (1992).

Monoclonal antibodies have been discovered which have agonist activity to the dimeric epidermal growth factor (EGF), tissue necrosis factor (TNF) and growth hormone (GRH) receptors. See Schreiber, A. B. et al., J. Biol. Chem. 258, 846-853 (1983), Defize, L. H. K. et al., EMBO J. 5, 1187-1992 (1986), Englemann, H. et al., J. Biol. Chem. 256, 14497-14504 (1990) and Fuh, G. et al., Science 260, 1808-1810 (1992). While not wishing to be bound to any particular theory of receptor activation, it is believed that in all three cases, the monoclonal antibodies, by virtue of possessing two antigen binding sites, were able to bridge two receptor molecules to facilitate aggregation and thus activate them.

Receptor-mediated biological functions are implicated in many conditions. Indications for compounds with agonist or antagonist activity towards single transmembrane receptors are numerous.

Despite the success of monoclonal antibodies in producing an agonist response in certain dimeric receptors, they are not considered ideal candidates for development of pharmaceutical compositions. Lack of oral bioavailability and a limited serum half-life limit the desirability and efficacy of monoclonal antibodies as pharmaceutical agents. Consequently, a need exists for non-antibody ligands which have agonist or antagonist properties towards dimeric or trimeric receptors.

### SUMMARY OF THE INVENTION

Accordingly, one aspect of the present invention is a method for agonizing or antagonizing a multimeric receptor comprising contacting the multimeric receptor with a non-antibody multimeric receptor ligand.

Another aspect of the invention is a method for identifying agonists and antagonists of multimeric receptors. The method comprises the steps of contacting a multimeric receptor with non-antibody multimeric receptor ligand candidates and selecting ligand candidates which bind to the receptor.

A third aspect of the invention is isolated non-antibody multimeric receptor agonists or antagonists.

A fourth aspect of the invention is a method for making non-antibody multimeric receptor ligands. The method comprises the steps of reacting a bifunctional monomer bound to a solid support with a receptor binding moiety and cleaving the reaction product from the solid support wherein the two functional groups are identical and symmetrically placed after cleavage.

### DETAILED DESCRIPTION OF THE INVENTION

Aspects of the present invention are non-antibody multimeric receptor agonists or antagonists and a method for agonizing or antagonizing a multimeric receptor by contacting the multimeric receptor with a non-antibody multimeric receptor ligand. A multimeric receptor is a receptor entity which is agonized or antagonized only when two or more subunits of the entity are aggregated on the same cell surface through binding to a common ligand.

Multimeric receptors which appear to signal by heterodimerization include granulocyte-macrophage-colony-stimulating factor (GM-CSF) receptor, the interleukins-3, -4, -5, -6, -12 and -13 (IL-3, 4, 5, 6, 12, and 13) receptors, oncostatin M, ciliary neurotropic factor (CNTF) receptor, leukemia inhibitory factor (LIF) receptor, nerve growth factor (NGF) receptor, fibroblast growth factor (FGF) receptor, the interferons α, β and γ (IFN-α, β and γ) receptors and TGF β1,2 receptor. Heterotrimeric signaling receptors include interleukin-2 (IL-2) receptor and tissue necrosis factor (TNF) receptor.

Known multimeric homodimerizing receptors include erythropoietin (EPO) receptor, granulocyte-colony-stimulating fctor (G-CSF) receptor, macrophage-colony-stimulating factor (M-CSF) receptor, tissue growth factor α (TGFα) receptor, epidermal growth factor (EGF) receptor, neu receptor, growth hormone (GRH) receptor, prolactin receptor, placental lactogen receptor, stem cell factor receptor (c-kit), tissue necrosis factor α and β (TNFα, β) receptors, fas receptor, CD40 receptor and CD27 receptor.

The non-antibody multimeric receptor ligand of the present invention serves as the common ligand through which two or more multimeric receptor subunits aggregate. The multimeric receptor ligand includes a spacer which is substituted with two or more receptor binding moieties.

The spacer can be any molecule having a di- or trisubstituted center capable of substitution with the receptor binding moieties. Preferably, the spacer provides for spatial separation and steric orientation of the receptor binding moieties which is sufficient to effectively induce aggregation while not sterically preventing such association. Most preferably, the spacer will provide spatial separation and steric orientation of the binding moieties which mimic the binding moieties of the natural ligand.

Exemplary disubstituted spacers include compounds represented by the formula (I):

—Z—(R)ₙ—(A)ₘ—(R)ₙ—Z— (I)

wherein:

A is independently N, O, S, dithio, carbonyl,

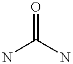

or nothing;

Z is independently N, O, S or carbonyl;

R is independently d- or 1-amino acid; alkyl of 1 to 10 carbons; cis, trans-2-butenyl; cis, trans-1,2-cyclopropyl; cis, trans-1,2-cyclobutyl; cis, trans-1,3-cyclobutyl; cis, trans-1,3-cyclopentyl; cis, trans-1,2-cyclopentyl; cis, trans-1,2-cyclohexyl; cis, trans-1,3-cyclohexyl; cis, trans-1,4-cyclohexyl; endo, exo-2,3-norbornane; 1,5-naphthyl; 2,6-naphthyl; 1,8-anthrylene; 1,5-anthrylene; 2,6-anthrylene;

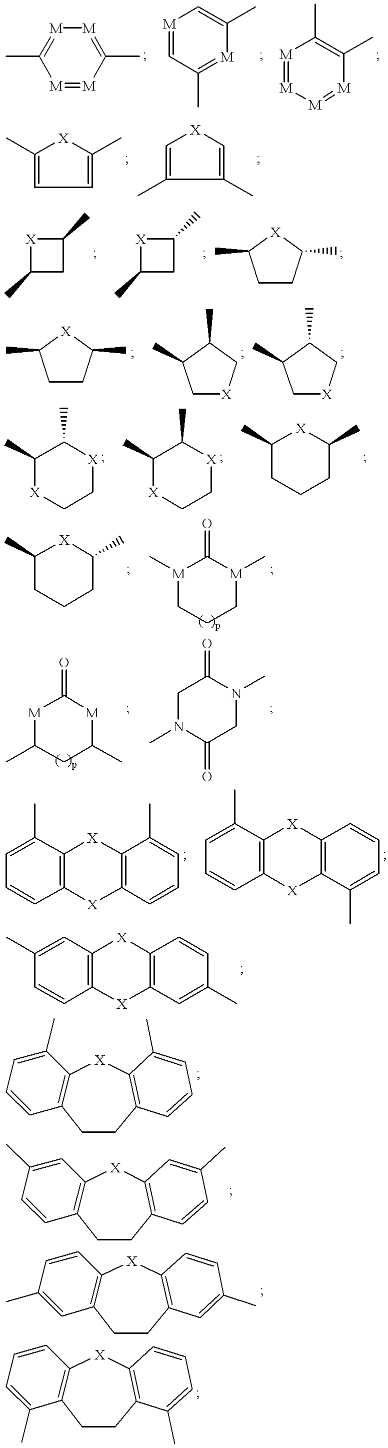

where

X is independently N, O or S;

M is independently C or N;

p is 0, 1, 2, or 3; and

m is 0 or 1; and

n is 0, 1, 2 or 3.

Preferred compounds of formula (I) are those where R is

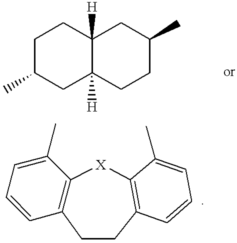

Exemplary trisubstituted spacers include compounds represented by the formula (II):

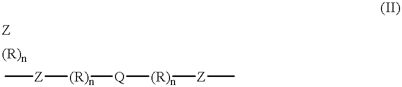

wherein:

Q is C; N; B; 1,3,5-phenyl; 1,3,5-cyclohexyl; 1,3,5-triazinyl;

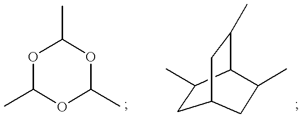

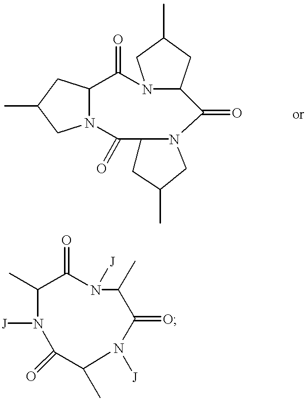

where J is independently H or alkyl of 1 to 10 carbons; and

Z is independently N, O, S or carbonyl;

R is independently d- or 1-amino acid; alkyl of 1 to 10 carbons; cis, trans-2-butenyl; cis, trans-1,2-cyclopropyl; cis, trans-1,2-cyclobutyl; cis, trans-1,3-cyclobutyl; cis, trans-1,3-cyclopentyl; cis, trans-1,2-cyclopentyl; cis, trans-1,2-cyclohexyl; cis, trans-1,3-cyclohexyl; cis, trans-1,4-cyclohexyl; endo, exo-2,3-norbornane; 1,5-naphthyl; 2,6-naphthyl; 1,8-anthrylene; 1,5-anthrylene; 2,6-anthrylene;

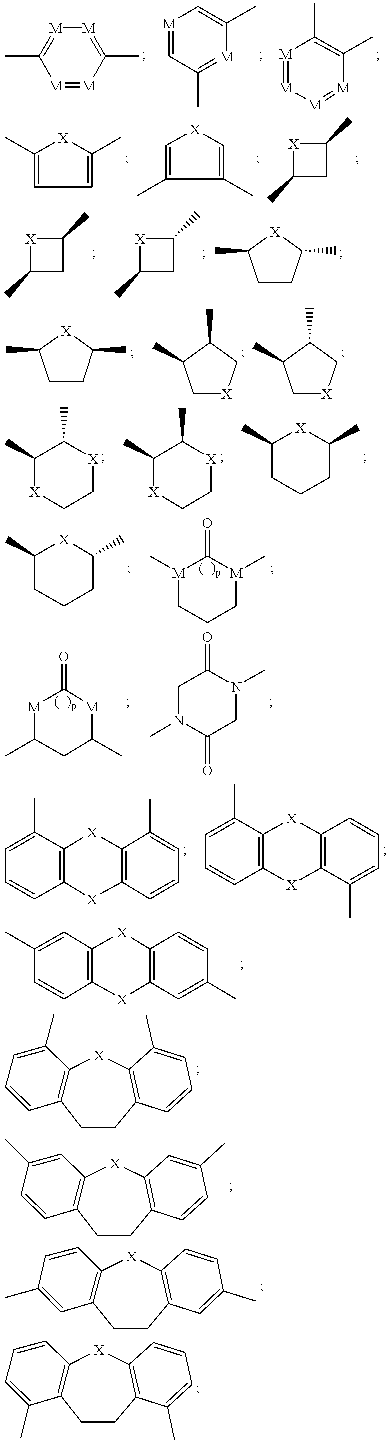

where

X is independently N, O or S;

M is independently C or N;

p is 0, 1, 2, or 3; and

m is 0 or 1; and

n is 0, 1, 2 or 3.

Preferred compounds of formula (II) are those where R is

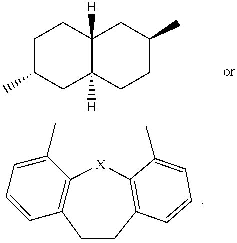

Also preferred are the compounds of formula (II) where Q is N; 1,3,5-phenyl and

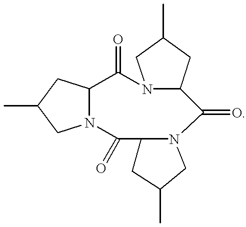

One skilled in the art could determine the required spatial separation and steric orientation of the receptor binding moieties through X-ray crystallographic data for receptor entities with and without bound natural ligand. For example, the X-ray crystal structures of HGH (a 4-helix bundle protein) complexed with its homodimeric receptor, HGH binding protein (HGHBP), have been published by Fuh et al. in Science 256, 1677-1680 (1992). The two HGHBP molecules of the receptor bind HGH with considerable C₂ symmetry. Analysis of the superimposition of the crystal structure of HGH.(HGHBP)² with an identical crystal structure, where the identical crystal structure has been rotated through a C₂ axis to maximize the overlap of the binding proteins, provides vectors for points of attachment for the receptor binding moieties. Design of spacer molecules which contain these vectors could be aided by conducting three-dimensional compound database searches using programs such as CAVEAT (Regents of the University of California) or SYBYL 3-D (Tripos Associates Inc.) This process could yield both C₂ symmetric and non-C₂ symmetric spacers.

The spacers derived from examination of HGH may be useful in designing ligands for other hematopoietic receptors, since it is known that dimeric receptor ligands share common structural features which lead to aggregation of the receptor subunits. It is expected that this method could also be generalized to other receptor-ligand complexes when their crystal structures become available.

The receptor binding moieties which attach to these spacers can be either peptides or small molecules from a natural or synthetic source. The peptide sequences could be chosen from but not limited to linear and cyclic sequences known to be important for binding of hematopoietic proteins to their receptors. Particularly interesting are those sequences found in helices one and four of the four α-helix bundle class of protein ligands, since these helices are important for binding and seem optimally situated for dimerization. Also, the helices, unlike the loop regions of these receptors, are very similar in orientation throughout this class of proteins. The identity of the possible small molecules could be chosen from but not limited to agonists and antagonists derived from database screens and peptide mimetics.

The receptor binding moieties of the non-antibody multimeric receptor ligands of the present invention can be identical, yielding homomultimeric compounds, or they can be different, yielding heteromultimeric compounds. In general, non-antibody multimeric receptor ligands could be synthesized by reacting a bifunctional monomer bound to a solid support with a receptor binding moiety to form a reaction product followed by cleaving the reaction product from the solid support, wherein the two functional groups are identical and symmetrically placed after cleavage.

Those of ordinary skill in this art would recognize that any single peptide or small organic molecule could be coupled to the spacer substitution centers to provide homomultimeric receptor ligand candidates.

Heteromultimeric compounds can be produced through combinatorial chemistry methods in which a library of compounds is synthesized. Combinatorial synthetic methods known to those skilled in the art can produce library members simultaneously as a mixture or individually. Diverse sets of biopolymers such as peptides containing naturally occurring and non-naturally occurring α- and β-amino acids, oligonucleotides and oligosaccharides as well as small organic molecules can be produced.

Linear peptide and oligonucleotide libraries can be produced by synthesis on a solid support, such as synthesis beads, followed by cleavage from their supports. Solution synthetic methods could also be employed.

Small organic molecule library members are built up on a core structure template. The core structure is derivatized through a series of synthetic steps to produce a library containing a discrete number of independently variable substituents, functional groups or structural elements. Reaction conditions are selected such that each derivatized core structure is different from the others. Methods for derivatizing core structures are disclosed in U.K. Patent Application No. 9325621.2, which is incorporated herein by reference.

Non-antibody heteromultimeric receptor ligand candidates are provided by substitution of library members onto the spacer. The spacer is coupled to a solid support such as a synthesis bead and the combinatorial library members are built out from the substitution centers present on the spacer. After library synthesis is complete, the resulting ligand candidates are cleaved from the support by techniques well known to those skilled in the art.

Another aspect of the present invention is a method for identifying agonists and antagonists of multimeric receptors and the multimeric receptor ligands identified thereby. In the method, a multimeric receptor is contacted with non-antibody multimeric receptor ligand candidates. Ligand candidates which bind to the multimeric receptor are selected by receptor binding assays well known to those skilled in the art.

In general, a target receptor in isolated, immobilized or cell-bound form is contacted with a plurality of receptor ligand candidates and those candidates which bind to and interact with the receptor are selected. Binding or interaction can be measured directly by using radioactively labeled ligand candidates or by measuring any second messenger effect resulting from the interaction or binding of the ligand candidate. Alternatively, the ligand candidates can be subjected to competitive binding assays in which a known receptor ligand, labeled preferably with an analytically detectable reagent, most prefereably radioactivity, is included with the ligand candidates and a candidate's ability to inhibit the binding of the labeled ligand is measured.

Positive multimeric receptor ligand binding candidates are screened for biological function by any one of the receptor function assays well known to those skilled in the art. It is expected that a positive ligand binding candidate will exhibit agonist or antagonist activity in receptor function assays.

Any agonist or antagonist compounds identified can be isolated by affinity chromatography. Other isolation techniques for multimeric small organic molecules include labeling the receptor binding moieties as they are being synthesized with coding agents such as oligonucleotides and peptides or tagging the moieties with structurally related molecules that can be analyzed by electron capture capillary gas chromatography.

A non-limiting specific competitive binding assay example follows.

### COMPETITIVE BINDING ASSAY EXAMPLE A

Tissue containing the appropriate target receptor is homogenized, filtered through cheesecloth and centrifuged at 1500×g for 10 minutes. Alternatively, cell membrane preparations from cells transfected or transformed with the target receptor gene may be employed. The supernatant is decanted and the pellet is resuspended in an appropriate incubation buffer, e.g., 75 mM Tris.HCl, pH 7.4 containing 12.5 mM MgCl₂ and 1.5 mM EDTA. Membranes equivalent to 100 μg protein are incubated with 50 pmol radiolabeled receptor ligand and an appropriate amount of the ligand binding candidate in a total volume of 500 μl for 1 hr at 37° C. The binding reaction is terminated by dilution with the addition of 5 ml of cold incubation buffer and the bound tracer is separated from free by filtration on Whatman GF/C filter paper. The filter paper is washed several times with cold incubation buffer and then counted to determine the amount of bound ligand. The presence of a competing ligand is evidenced by a reduction in binding of the radiolabeled receptor ligand relative to a control lacking the addition of ligand binding candidate.

The present invention will now be described with reference to the following specific, non-limiting Examples 1 and 2. The diacids produced by the methods of Examples 1 and 2 and other diacids within the scope of the invention can serve as a spacer by attachment through an amide or ester bond. They can also be reduced to the corresponding alcohols using a reducing agent such as borane, LiAlH₄ or diisobutylaluminum hydride; this alcohol can be converted to a leaving group using mesyl chloride, triphenylphosphine and CCl₄ or tosyl chloride. This leaving group can be used to attach the linker to the binding moieties through an ether, amine, sulfide or hydrocarbon linkage. The diamines produced can be attached to the binding moieties through an amide, urea, carbamate or amine linkage. These diacids and diamines can be elaborated further to create other linkers or attached to a resin used in creating combinatorial libraries.

### EXAMPLE 1

### Synthesis of the Disubstituted Spacer 4,6-Dicarboxyiminodibenzyl and 4,6-Diaminoiminodibenzyl

The synthetic steps are outlined in Scheme 1 below.

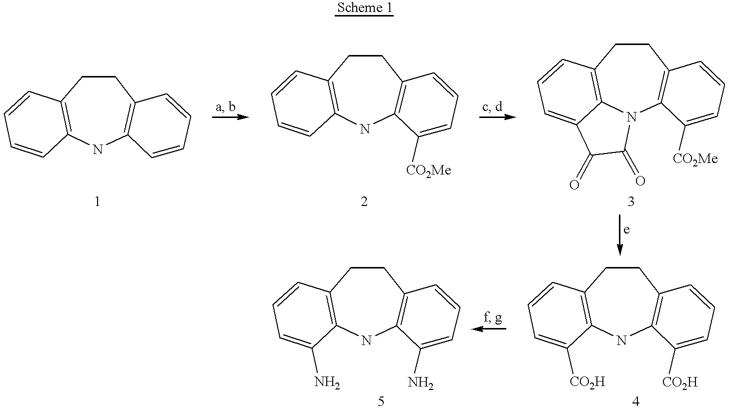

a) n-butyllithium, CO₂; b) CH₂N₂, ethyl ether; c) ClCOCOCl, ethyl ether; d) CS₂, AlCl₃; e) H₂O₂, OH—; f) diphenylphosphorylazide, triethylamine, t-butanol; g) trifluoroacetic acid

The monoester 2 is available in two steps from iminodibenzyl 1 (available from Aldrich Chemical Co., Milwaukee, Wis.). The iminodibenzyl is first dilithiated with two equivalents of an alkyl lithium, such as n-butyllithium, to form the dianion which is subsequently treated with carbon dioxide to form the carboxylic acid. This monocarboxylic acid can then be esterified by standard techniques, such as diazomethane in ether. The monoester 2 can be acylated at the 4-position by a two step procedure. The iminodibenzyl 2 is first treated with oxalyl chloride to form the amide; this intermediate cyclizes to the a-ketoamide upon treatment with a Lewis acid such as AlCl₃, TiCl₄ or FeCl₃. The α-ketoamide 3 can be converted to the diacid by treatment with an oxidizing agent such as H₂O₂ or NaIO₄ and hydrolysis with hydroxide anion (Hess, B. A. et al. J. Am. Chem. Soc. 91, 1672 (1969)). The diacid can also be converted to the diamine using standard Curtius conditions (diphenylphosphorylazide and triethylamine or NaN₃ and ClCOCOCl) and the t-butyl carbamate produced can be hydrolyzed with trifluoroacetic acid.

### EXAMPLE 2

### Synthesis of the Disubstituted Spacer trans-2,6-Dicarboxydecalin and trans-2,6-Diaminodecalin

The synthetic steps are outlined in Scheme 2 below.

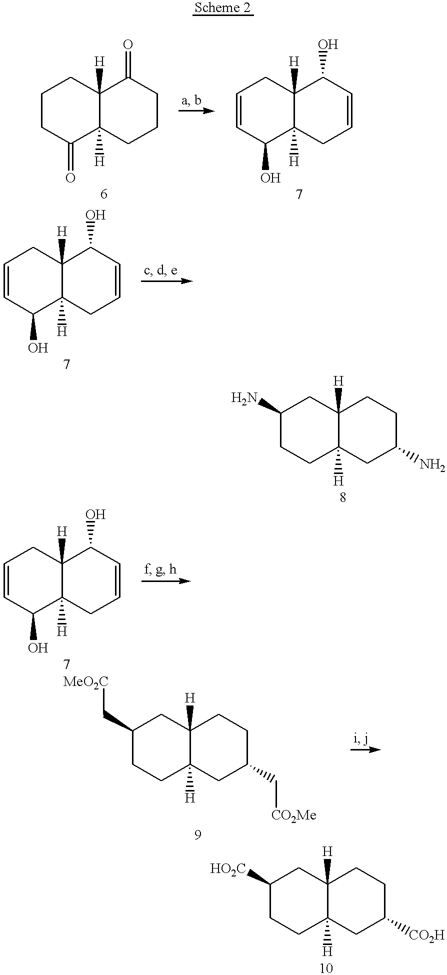

a) i) lithium diisopropylamide, phenylselenenyl chloride; ii) m-chloroperoxybenzoic acid, triethylamine; b) lithium tri-sec-butylborohydride; c) Cl₃CCN, heat; d) H₂, Pd; e) NaOH; f) CH₃O₂CCl, pyridine; g) Tebbe reagent, heat; h) H₂, Pd; i) lithium diisopropylamide, O₃; j) AgO

The diol 7 is synthesized in two steps from trans-1,5-decalindione (available from Aldrich Chemical Co., Milwaukee, Wis.). The first-step conversion to the α,β-unsaturated ketones involves conversion to the α-phenyl selenide using a sequential addition of a strong base, such as lithium diisopropylamide or bis[trimethylsilyl]amide, and phenylselenenyl chloride, oxidation of the selenium using hydrogen peroxide or m-chloroperoxybenzoic acid followed by a quench of the oxidant and basic elimination. The α,β-unsaturated ketones are then reduced to the corresponding axial alcohols using a bulky hydride, such as L-Selectride® (1.0 M lithium tri-sec-butylborohydride in tetrahydrofuran) or K-Selectride® (1.0 M potassium tri-sec-butylborohydride in tetrahydrofuran), which prefer equatorial attack. Intermediate 7 is then converted to the desired diamine by conversion to the corresponding trichloroimidate followed by rearrangement to the transposed allylic amide (Overman, L. J. Am. Chem. Soc. 98, 2901-2910 (1976)). The allylic amide is then reduced to the amide by hydrogenation using Pd, Wilkinson's catalyst (tris[triphenylphosphine]rhodium[1]chloride) or Pt as a catalyst and the amide is hydrolyzed using basic hydrolysis such as NaOH, KOH or LiOOH to form the diamine 8. The diol 7 can also be converted to the diacid 10 in five steps. The carbonate is formed using methylchloroformate and a base such as pyridine or triethylamine. The carbonate is then methylenated using Tebbe reagent and the enol ether undergoes an allylic rearrangement. The ester is then hydrogenated using Pd, Wilkinson's catalyst or Pt as a catalyst to form 9. The ester is then enolized using a strong base such as lithium diisopropylamide or sodium bis[trimethylsilyl]amide and the enolate is cleaved with ozone. The dialdehyde produced is treated with silver oxide to form the diacid.

The present invention may be embodied in other specific forms without departing from the spirit or essential attributes thereof and, accordingly, reference should be made to the appended claims, rather than to the foregoing specification, as indicating the scope of the invention.

1. A method for agonizing or antagonizing a multimeric receptor comprising contacting the multimeric receptor with a non-antibody multimeric receptor ligand.

2. The method of claim 1 wherein the multimeric receptor is a dimeric receptor.

3. The method of claim 2 wherein the dimeric receptor is homodimeric.

4. The method of claim 3 wherein the non-antibody multimeric receptor ligand is homodimeric.

5. The method of claim 2 wherein the dimeric receptor is heterodimeric.

6. The method of claim 5 wherein the non-antibody multimeric receptor ligand is heterodimeric.

7. The method of claim 2 wherein the dimeric receptor is a hematopoietic growth factor receptor.

8. The method of claim 2 wherein the dimeric receptor is erythropoietin receptor, granulocyte-colony-stimulating factor receptor, macrophage-colony stimulating factor receptor, tissue growth factor α receptor, epidermal growth factor receptor, neu receptor, growth hormone receptor, prolactin receptor, placental lactogen receptor, stem cell factor receptor, tissue necrosis factor α receptor, tissue necrosis factor β receptor, fas receptor, CD40 receptor or CD27 receptor.

9. The method of claim 2 wherein the dimeric receptor is platelet-derived growth factor receptor, insulin receptor, insulin-like growth factor-l receptor, insulin-like growth factor-2 receptor or relaxin receptor.

10. The method of claim 2 wherein the dimeric receptor is granulocyte-macrophage colony stimulating factor receptor, interleukin-3 receptor, interleukin-5 receptor, interleukin-6 receptor, oncostatin M receptor, ciliary neurotropic factor receptor, leukemia inhibitory factor receptor, nerve growth factor receptor, fibroblast growth factor receptor, interleukin-4 receptor, interleukin-13 receptor, interferon α receptor, interferon β receptor, interferon γ receptor, TGF β1,2 receptor or interleukin-12 receptor.

11. The method of claim 1 wherein the multimeric receptor is a trimeric receptor.

12. The method of claim 11 wherein the trimeric receptor is heterotrimeric.

13. The method of claim 12 wherein the trimeric receptor is interleukin-2 receptor.

14. The method of claim 12 wherein the trimeric receptor is tissue necrosis factor receptor.

15. A method for identifying agonists and antagonists of multimeric receptors comprising the steps of:
a) contacting a multimeric receptor with non-antibody multimeric receptor ligand candidates; and
b) selecting ligand candidates which bind to the receptor.

16. Multimeric receptor ligands identified by the method of claim 15.

17. Isolated non-antibody multimeric receptor agonists.

18. Isolated non-antibody multimeric receptor antagonists.

19. The isolated non-antibody multimeric receptor agonists or antagonists of claims 17 or 18 comprising a disubstituted spacer having the formula (I):
—Z—(R)ₙ—(A)ₘ—(R)ₙ—Z— (I)
wherein:
A is independently N, O, S, dithio, carbonyl, 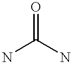
or nothing;
Z is independently N, O, S or carbonyl;
R is independently d- or 1-amino acid; alkyl of 1 to 10 carbons; cis, trans-2-butenyl; cis, trans-1,2-cyclopropyl; cis, trans-1,2-cyclobutyl; cis, trans-1,3-cyclobutyl; cis, trans-1, 3-cyclopentyl; cis, trans-1, 2-cyclopentyl; cis, trans-1,2-cyclohexyl; cis, trans-1,3-cyclohexyl; cis, trans-1, 4-cyclohexyl; endo, exo-2,3-norbornane; 1,5-naphthyl; 2,6-naphthyl; 1,8-anthrylene; 1,5-anthrylene; 2,6-anthrylene; 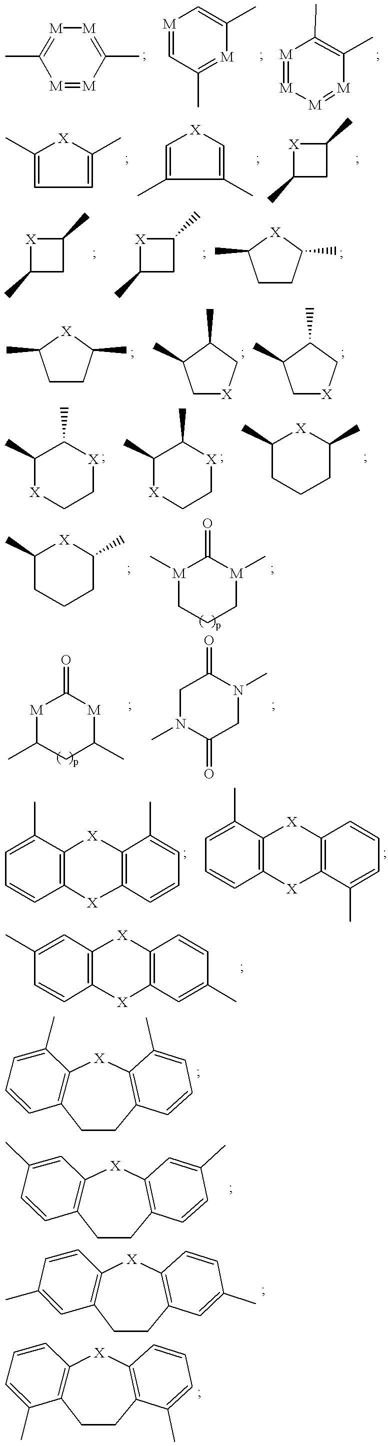
where
X is independently N, O or S;
M is independently C or N;
p is 0, 1, 2, or 3; and
m is 0 or 1; and
n is 0, 1, 2 or 3.

20. The isolated non-antibody multimeric receptor agonists or antagonists of claim 19 wherein R is

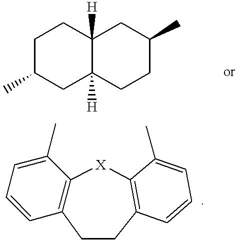

21. The isolated non-antibody multimeric receptor agonists or antagonists of claims 17 or 18 comprising a trisubstituted spacer having the formula (II): 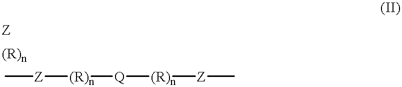
wherein:
Q is C; N; B; 1,3,5-phenyl; 1,3,5-cyclohexyl; 1,3,5-triazinyl; 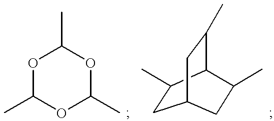 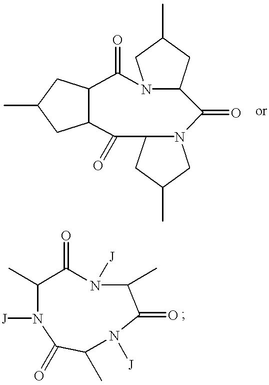
where J is independently H or alkyl of 1 to 10 carbons; and
Z is independently N, O, S or carbonyl;
R is independently d- or 1-amino acid; alkyl of 1 to 10 carbons; cis, trans-2-butenyl; cis, trans-1, 2-cyclopropyl; cis, trans-1,2-cyclobutyl; cis, trans-1,3-cyclobutyl; cis, trans-1,3-cyclopentyl; cis, trans-1,2-cyclopentyl; cis, trans-1,2-cyclohexyl; cis, trans-1,3-cyclohexyl; cis, trans-1,4-cyclohexyl; endo, exo-2,3-norbornane; 1,5-naphthyl; 2,6-naphthyl; 1,8-anthrylene; 1,5-anthrylene; 2,6-anthrylene; 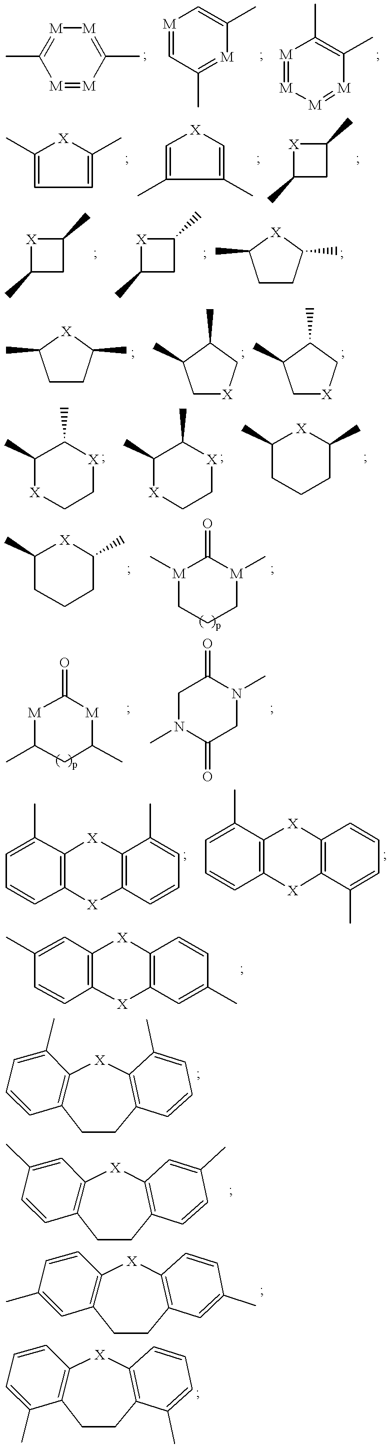
where
X is independently N, O or S;
M is independently C or N;
p is 0, 1, 2, or 3; and
m is 0 or 1; and
n is 0, 1, 2 or 3.

22. The isolated non-antibody multimeric receptor agonists or antagonists of claim 21 wherein R is

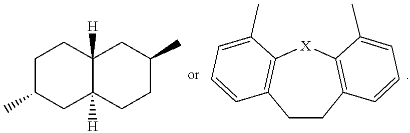

23. The isolated non-antibody multimeric receptor agonists or antagonists of claim 21 wherein Q is N; 1,3,5-phenyl and

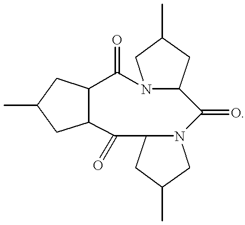

24. A method for making non-antibody multimeric receptor ligands comprising the steps of:
a) reacting a bifunctional monomer bound to a solid support with a receptor binding moiety to form a reaction product; and
b) cleaving the reaction product from the solid support,
wherein the two functional groups are identical and symmetrically placed after cleavage.
